(19) United States
(12) Patent Application Publication
(10) Pub. No.: US 2004/0092527 A1
(43) Pub. Date: May 13, 2004

(54) Itraconazole bioavailability

(76) Inventors: Ramaswami Bharatrajan, Maharashtra (IN); Deepak Hegde, Maharashtra (IN); Neeta Nerlekar, Maharashtra (IN)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/311,943

(22) PCT Filed: Jun. 19, 2001
(86) PCT No.: PCT/EP01/06917

(30) Foreign Application Priority Data
Jun. 21, 2000 (GB) 0015239.7

(51) Int. Cl. A61K 31/724; A61K 31/496

(52) U.S. Cl. 514/254.07; 514/58

(57) ABSTRACT

A composition comprising itraconazole and an organic acid and the use of such composition in the production of pharmaceutical compositions comprising itryconazole as an active ingredient.

The present invention relates to the improvement of bioavailability of the antifungal compound itraconazole, see e.g. Merck Index, 12^{th} edition, pages 894-895, item 5262, e.g. active in the treatment of candidiasis, tinea, pityriasis, versicolor and systemic fungal infections. Itroconazole may be e.g. administered orally, e.g. at a daily dose of 100 mg to 400 mg.

Bioavailability of itraconazole may be restricted in aqueous media, e.g. because itraconazole is known to be insoluble in water and in acidic, e.g. aqueous, e.g. diluted hydrochloric acidic; solvent; e.g. under conditions as present in the stomach. In WO00/00179 a solid dispersed preparation for poorly water-soluble drugs, prepared by dissolving or dispersing the drug in an oil, a fatty acid or a mixture thereof, mixing the solution or dispersion in a water-soluble polyol matrix and drying the mixture is described which preparation allegedly allows poorly water-soluble drugs to be increased in the uptake efficiency in the gastro-intestinal tract.

In WO94/16733 multicomponent inclusion complexes characterized in that a basic-type drug (guest molecule), e.g. itraconazole, an acid and a cyclodextrin are present, are described. Allegedly the presence of an acid in the formation of complexes of amine type drugs with cyclodextrins results in easily water-soluble complexes.

In WO98/55148 pharmaceutical compositions comprising a no more than sparingly water-soluble drug compound, a cyclodextrin, a physiologically tolerable water-soluble acid, and a physiologically tolerable water-soluble organic polymer, are described. Allegedly thus an administration form can be produced which improves the biological uptake of the drug compound.

In WO95/08993 formulations for oral administration comprising an antifungal, a sufficient amount of a cyclodextrin or a derivative thereof, an aqueous acidic medium as bulk liquid carrier and an alcoholic co-solvent; are described.

In WO00/07596 pharmaceutical water-soluble formulations with a content on the phosphodiesterase (PDE)-type-5-inhibitor sildenafil or pharmaceutically acceptable salts thereof, optionally in combination with an CYP-inhibitor as erythromycin, cimetidine, ketoconazole, itroconazole or mibefradil and optionally comprising citric, ascorbic and/or tartaric acid; are described.

It was now surprisingly found that the solubility and in consequence the bioavailability of itraconazole in diluted hydrochloric acidic aqueous solvent may be improved by simple combination of itraconazole with an organic acid.

In one aspect the present invention provides a composition comprising itraconazole and an organic acid with the proviso that

formulations for oral administration comprising an antifungal, a sufficient amount of a cyclodextrin or a derivative thereof, an aqueous acidic medium as a bulk liquid carrier and an alcoholic co-solvent,

multicomponent inclusion complexes characterized in that a basic-type drug (guest molecule), an acid and a cyclodextrin are present,

solid dispersed preparations for poorly water-soluble drugs, prepared by dissolving or dispersing the drug in anoil, a fatty acid or a mixture thereof, mixing the solution or dispersion in a water-soluble polyol matrix and drying the mixture,

pharmaceutical compositions comprising a no more than sparingly water-soluble drug compound, a cyclodextin, a physiologically tolerable water-soluble acid, and a physiologically tolerable water-soluble organic polymer, and

pharmaceutical water-soluble formulations with a content on the phosphodiesterase (PDE)-type-5-inhibitor sildenafil or pharmaceutically acceptable salts thereof in combination with itraconazole and comprising citric, ascorbic and/or tartaric acid; are excluded.

A composition according to the present invention is preferably a pharmaceutically acceptable composition. The composition according to the present invention may consist essentially of itraconazole and an organic acid, or it may further contain pharmaceutically acceptable excipients, e.g. excipient(s) which is (are) appropriate in a pharmaceutical composition.

In a composition according to the present invention the ratio of itraconazole and organic acid is not critical. Per equivalent of itraconazole one equivalent or less than one equivalent or more than one equivalent of an organic acid may be present.

Appropriate organic acids includes e.g. a pharmaceutically acceptable organic acids, such as carboxylic acids and sulfonic acids, preferably carboxylic acids. Appropriate organic acids include organic acids having more than one acid function, e.g. dicarboxylic acids and disulfonic acids. Appropriate carboxylic acids include e.g. (C₃₋₂₃)carboxylic acids (including the carbon atom of the carboxylic group(s)), e.g. including saturated and unsaturated carboxylic acids, e.g. including unsubstituted and substituted carboxylic acids, e.g. unsubstituted carboxylic acids or substituted by hydroxy; such as aliphatic carboxylic acids, e.g. saturated aliphatic carboxylic acids, such as propionic acid, citric acid, lactic acid; tartaric acid; and unsaturated aliphatic carboxylic acids, e.g. including fatty acids; such as maleic acid, fumaric acid, succinic acid, myristic acid. Appropriate organic acids include mixtures of individual carboxylic acids; e.g. such as cited above. Preferred organic carboxylic acids include e.g. (C₃₋₈)aliphatic carboxylic acids, most preferably succinic acid or maleic acid. Sulfonic acids include e.g. aliphatic and aromatic sulfonic acids, such as alkylsulfonic acids, e.g. including (C₁₋₆)alkylsulfonic acids, e.g. methanesulfonic acid, ethanesulfonic acid; and (C₆₋₁₂)aromatic sulfonic acids, such as benzenesulfonic acid, p-toluenesulfonic acid, naphthalenesulfonic acids.

It was surprisingly found that in a composition according to the present invention itraconazole may be present in a form wherein chemical and/or physical characteristics of itraconazole are different from chemical and/or physical characteristics of pure itraconazole. For example, in a composition according to the present invention comprising itraconazole and succinic acid the Raman spectrum (FIG. 3), the X-ray powder diffraction pattern diagram (FIG. 2) and the melting point of itraconazole in said composition are different from those of pure itraconazole. It is thus believed that itraconazole in a composition according to the present invention is in a complexed form of itraconazole with an organic acid which consitutes a novel chemical entity.

A composition comprising, e.g. consisting essentially of, itraconazole and an organic acid wherein chemical and/or physical characteristics of itraconazole in said composition are different from chemical and/or physical characteristics, e.g. itraconazole in the form of a complex with an organic acid is hereinafter designated as “Complexed itraconazole”. Complexed itraconazole may have a higher solubility in water or (diluted) acidic aqueous solvent than itraconazole, e.g. as shown in FIG. 1.

The mol ratio of itraconazole and a carboxylic acid in a composition comprising itraconazole and an organic acid wherein chemical and/or physical characteristics of itraconazole in said composition are different from chemical and/or physical characteristics of pure itraconazole, e.g. wherein itraconazole is in the form of a complex; is not critical. If it is desired to obtain only a part of complexed itraconazole in a composition according to the present invention (the other part being uncomplexed itraconazole), per equivalent of itraconazole less than one equivalent of organic acid may be present in a composition according to the present invention. If it is desired to obtain the whole amount of itraconazole present in a composition according to the present invention in the form of complexed itraconazole at least one equivalent or more of an organic acid may present. Per equivalent of itraconazole:preferably 0.1 to 10, such as 0.5 to 5, e.g. 1 to 2.5 equivalents of an organic acid may be appropriate.

In another aspect the present invention provides a composition comprising itraconazole and an organic acid, wherein chemical and/or physical characteristics of itraconazole are different from chemical and/or physical characteristics of pure itraconazole; and in another aspect

Itraconazole in the form of a complex with an organic acid.

Chemical and/or physical characteristics of itraconazole which may be different in a composition according to the present invention in respect with pure itraconazole include e.g. the X-ray-powder diffraction pattern diagram, the Ramanspectrum, the IR-spectrum, and/or the melting point.

A composition, e.g. complexed itraconazole; according to the present invention may further comprise at least one excipient; e.g. excipients which are pharmaceutically acceptable. “Excipient(s)” as used herein are auxiliaries in the preparation of pharmaceutical compositions. Appropriate excipients include excipients according to known useful excipients in the production of pharmaceutical, e.g. oral; compositions, e.g. including carrier or diluent; useful in the production of pharmaceutical compositions, e.g. preferably oral pharmaceutical compositions, such as dosage forms; e.g. including tablets, e.g. film-coated tablets; capsules, e.g. hard gelatine capsules; powders and granules, e.g. useful as such or in the production of sirups, e.g. by reconstitution of powders, granules in a liquid, e.g. an aqueous liquid, such as water.

In another aspect the present invention provides a composition comprising itraconazole and an organic acid, e.g. complexed itraconazole, and further comprising at least one excipient.

An excipient present in a composition according to the present invention preferably includes one or more, e.g. at least one; pharmaceutically acceptable excipients useful in the production of pharmaceutical compositions, e.g.

surfactant; e.g. including anionic, cationic and non-ionic; preferably non-ionic, polymers, such as polyoxyethylene-polyoxypropylene-copolymers (blockpolymers), e.g. Poloxamer(s)®, Pluronic(s)®;

hydrocolloid; e.g. including povidone, celluloses, such as ethylcelluloses, hydroxypropylcelluloses, hydroxypropylmethylcelluloses;

buffer substance; e.g. potassium dihydrogen phosphate;

filler, e.g. including lactose, mannit, cellulose(s);

lubricant, e.g. including stearic acid, magnesium stearate;

binder, e.g. including microcrystalline cellulose, such as Avicel(s)®;

disintegrant, e.g. croscarmellose sodium;

flavour; e.g. including pharmaceutically acceptable flavour sweetener; e.g. including saccharine, e.g. sodium; cyclamate, aspartame.

A composition, e.g. complexed itraconazole; according to the present invention may e.g. be produced by

mechanical treatment of itraconazole and an organic acid, e.g. a mixture of itraconazole and an organic acid may be prepared by, e.g. intensive, grinding. milling, compacting;

heat treating, e.g. melting, a mixture of itraconazole and an organic acid;

co-precipitating, e.g. including crystallisation, of itraconazole and an organic acid from a solution/suspension in organic solvent; e.g. including mixtures of individual solvents, e.g. by evaporation, addition of anti-solvent (solvent wherein the co-precipitate is less soluble than in the solvent used for the preparation of the solution/suspension); or

spray-drying of a solution/suspension of itraconazole and an organic acid in appropriate solvent.

Excipient(s) may be added before any treatment and/or after any treatment.

In another aspect the present invention provides a process for the production of a composition comprising, e.g. consisting essentially of; itraconazole and an organic acid, e.g. complexed itraconazole; e.g. further comprising at least one excipient, which process comprises

mechanical treatment of itraconazole and an organic acid; e.g. optionally comprising at least one excipient and/or optionally adding at least one excipient after said mechanical treatment to the mixture obtained;

melting itraconazole and an organic acid; e.g. optionally comprising at least one excipient and/or optionally adding at least one excipient after said melting to the product obtained; e.g. optionally after grinding, milling;

crystallising a composition according to the present invention from a solution comprising itraconazole and an organic acid in appropriate solvent; e.g. and optionally adding at least one excipient to crystalls thus obtained, e.g. after grinding milling;

co-precipitating a composition according to the present invention from appropriate solvent; e.g. said composition optionally comprises at least one excipient and/or optionally adding at least one excipient after said co-precipitation to the co-precipitate obtained;

spray-drying a solution or suspension of itraconazole and an organic acid in appropriate solvent, e.g. said solution or suspension optionally comprises at least one excipient and/or optionally adding at least one excipient to the spray dried solid obtained.

In a preferred embodiment of the present invention a process for the production of a composition according to the present invention may be carried out as set out below:

itraconazole and an organic acid, e.g. optionally comprising at least one excipient, may be mixed and the mixture may be grinded together; e.g. by use of a mill; and the grinded mixture obtained may undergo thermal treatment, e.g. may be heated; e.g. the mixture may be melted, and cooled, e.g. and the residue obtained may be diminished, e.g. by grinding; e.g. or the residue obtained may be re-crystallized; in appropriate solvent; e.g. in organic solvent (mixtures), e.g. a mixture of methanol and dichloromethane; e.g. and at least one excipient may be added optionally;

itraconazole and an organic acid; e.g. optionally comprising at least one excipient, may be melted and the melt obtained may be cooled; e.g. and, if desired, the residue obtained upon cooling may be optionally diminished, e.g. by grinding, milling; e.g. and may be optionally recrystallized; in appropriate solvent; e.g. in organic solvent (mixtures), e.g. a mixture of methanol and dichloromethane; e.g. and optionally at least one excipient may be added;

itraconazole and an organic acid; e.g. optionally comprising at least one excipient, may be dissolved in appropriate solvent, e.g. which may easily be found, e.g. by pre-testing: e.g. and which includes organic solvent (mixtures), e.g. a mixture of methanol and dichloromethane; and from the solution obtained a composition, e.g. itraconazole in complexed form, may be crystallized; e.g. and the crystalls obtained may be isolated; e.g. and optionally at least one excipient may be added to the isolated crystalls, e.g. optionally after grinding, milling;

a mixture of itraconazole and an organic acid; e.g. optionally comprising at least one excipient, may be dissolved in appropriate solvent, e.g. which may easily be found, e.g. by pre-testing: e.g. and which includes organic solvent (mixtures), e.g. a mixture of methanol and dichloromethane; and from the solution obtained a composition, e.g. itraconazole in complexed form, e.g. comprising at least one excipient, may be co-precipitated; e.g. by solvent removal, e.g. including distillation such as evaporation; addition of anti-solvent; and the co-precipitate obtained may be isolated; e.g. and at least one excipient may be added optionally to the isolated precipitate;

a mixture of itraconazole and an organic acid; e.g. optionally comprising at least one excipient, may be dissolved or suspended in appropriate solvent, e.g. which may easily be found, e.g. by pre-testing: e.g. and which includes organic solvent (mixtures), e.g. a mixture of methanol and dichloromethane; and the mixture or suspension obtained may be spray-dried; e.g. according to an appropriate method, such as a method as conventional; e.g. and at least one excipient may be added optionally and mixed into the spray dried residue.

A composition, e.g. complexed itraconazole; according to the present invention comprising at least one excipient; may be obtained.

It was surprisingly found, that a composition, e.g. complexed itraconazole; according to the present invention, e.g. optionally comprising at least one excipient, such as a hydrocolloid, e.g. hydroxpropylcellulose, a surfactant, e.g. a polyoxyethylene-polyoxypropylenecopolymer, a lubricant, e.g. magnesium stearate, a filler, e.g. lactose; e.g. and potassium dihydrogen phophate; may have an in vitro dissolution rate which is after 5 minutes 2 times and more, e.g. about 2 to 3 times, after 10 minutes about 2 times and more, e.g. about 2 to 3 times; and after 15 minutes about two times faster than the dissolution rate of a commercially available itraconazole composition; e.g. Sporanox® 100 mg capsules, Janssen Cilag B.V., #99B08/175) or similar formulations; in the USP paddle test, 75 rpm, 1000 ml 0.1N HCl+0.2% SLS.

In another aspect the present invention provides a composition, e.g. complexed itraconazole; according to the present invention, wherein itraconazole has a dissolution rate in the USP paddle test, 75 rpm, 1000 ml 0.1N HCl+0.2% SLS, UV detection at 258 nm; which is after 10 minutes faster, e.g. 2 to 3 times faster; than the dissolution rate of itraconazole in Sporanox® formulations, e.g. a dissolution rate which is

after 5 minutes of about 55% to 65%, such as around 60%; and/or

after 10 minutes of about 75% to 85%, e.g. around 80%; and/or;

after 15 minutes of about 86% to 92%, e.g. around 90%.

A composition, e.g. complexed itraconazole; according to the present invention, comprising at least one excipient, e.g. a carrier or diluent, is useful in the production of pharmaceutical, e.g. oral; compositions, such as dosage forms; e.g. including tablets, e.g. film-coated tablets; capsules, e.g. hard gelatine capsules; powders and granules, e.g. powders or granules useful as such; or useful in the production of orally administrable sirups, e.g. sirups reconstituted from powders or granules in a liquid, e.g. aqueous liquid, such as water.

In another aspect the present invention provides the use of a composition of the present invention in the production of a pharmaceutical composition comprising itraconazole as an active ingredient.

Tablets, film-coated tablets, powders and granules according to the present invention may be produced as appropriate, e.g. according to a method as conventional; e.g. tablets may be produced by compressing a composition according to the present invention, e.g. complexed itraconazole, e.g. comprising appropriate excipients, e.g. excipients useful in the production of tablets; e.g. excipients according to excipients as conventional in tabletting processes; e.g. and film-coating as appropriate; e.g. according to a method as conventional. Capsules may be filled with a composition according to the present invention, e.g. complexed itraconazole, e.g. comprising excipients useful; e.g. excipients as conventional; in a composition administered in the form of a capsule. Powders may be produced e.g. by grinding or spray-drying of a composition according to the present invention, e.g. complexed itraconazole; e.g. comprising excipients; e.g. according to excipients as conventional; useful in the production of powders. And granules may be produced by appropriate granulation techniques; e.g. according to a method as conventional.

In another aspect the present invention provides a pharmaceutical composition comprising itraconazole and a pharmaceutically acceptable organic acid beside at least one pharmaceutical carrier or diluent with the proviso that

formulations for oral administration comprising an antifungal, a sufficient amount of a cyclodextrin or a derivative thereof, an aqueous acidic medium as a bulk liquid carrier and an alcoholic co-solvent,

multicomponent inclusion complexes characterized in that a basic-type drug (guest molecule), an acid and a cyclodextrin are present,

solid dispersed preparations for poorly water-soluble drugs, prepared by dissolving or dispersing the drug in an oil, a fatty acid or a mixture thereof, mixing the solution or dispersion in a water-soluble polyol matrix and drying the mixture,

pharmaceutical compositions comprising a no more than sparingly water-soluble drug compound, a cyclodextin, a physiologically tolerable water-soluble acid, and a physiologically tolerable water-soluble organic polymer, and

pharmaceutical water-soluble formulations with a content on the phosphodiesterase (PDE)-type-5-inhibitor sildenafil or pharmaceutically acceptable salts thereof in combination with itraconazole and comprising citric, ascorbic and/or tartaric acid; are excluded.

In another aspect the present invention provides a pharmaceutical composition according to the present invention wherein the pharmaceutical carrier or diluent comprises lubricant, and/or a binder, and/or filler, and or disintegrant, and/or surfactant; and/or hydrocolloid, and/or buffer substance.

In another aspect the present invention provides a pharmaceutical composition comprising, e.g. consisting essentially of, itraconazole, an organic acid, e.g. maleic acid and a lubricant, e.g. magnesium stearate, e.g. filled into hard gelatine capsules.

In another aspect the present invention provides a pharmaceutical composition comprising, e.g. consisting essentially of, itraconazole, an organic acid, e.g. maleic acid, binder, e.g. microcrystalline cellulose, filler, e.g. lactose, disintegrant, e.g. croscarmellose sodium, and lubricant, e.g. magnesium stearate, e.g. compressed into tablets.

In another aspect the present invention provides a pharmaceutical composition comprising, e.g. consisting essentially of, itraconazole, organic acid, e.g. succinic acid, surfactant, e.g. a copolymer of a polyoxyethylene-polyoxypropylene-blockpolymer, hydrocolloid, e.g. hydroxypropylmethylcellulose, buffer substance, e.g. potassium dihydrogen phosphate, filler, e.g. lactose and lubricant, e.g. magnesium stearate, e.g. filled into hard gelatine capsules.

In another aspect the present invention provides a pharmaceutical composition comprising, e.g. consisting essentially of, itraconazole, organic acid, e.g. succinic acid, surfactant, e.g. a copolymer of a polyoxyethylene-polyoxypropylene-blockpolymer, filler, e.g. lactose and lubricant, e.g. magnesium stearate, e.g. filled into hard gelatine capsules.

In another aspect the present invention provides a pharmaceutical composition comprising, e.g. consisting essentially of, itraconazole, organic acid, e.g. succinic acid, hydrocolloid, e.g. hydroxypropylmethylcellulose, buffer substance, e.g. potassium dihydrogen phosphate, filler, e.g. lactose and lubricant, e.g. magnesium stearate, e.g. filled into hard gelatine capsules.

A pharmaceutical composition comprising itraconazole and a pharmaceutically acceptable carboxylic acid beside at least one pharmaceutical carrier or diluent may be used for the same indications in the same dosage ranges as itraconazole in a pharmaceutical composition as commercially available, e.g. Sporanox® or similar pharmaceutical compositions. A pharmaceutical composition according to the present invention may have a better dissolution rate and a better bioavailability of itroconazole than a pharmaceutical composition which is currently commercially available e.g. Sporanox® or similar pharmaceutical compositions.

In the following examples all temperatures are given in degree Celsius.

### EXAMPLE 1

Complexed Itraconazole in Capsules

8.6 g of itraconazole and 1.4 g of maleic acid are mixed and grinded. The mixture obtained is heated until a melt is formed. The melt obtained is cooled to room temperature, grinded in a mill and blended with magnesium stearate.

The mixture obtained is filled into hard gelatine capsules.

### EXAMPLE 2

Complexed Itraconazole in Tablet Form

A grinded melt is formed according to the method of example 1. The grinded melt is blended with microcrystalline cellulose (Avicel 101), lactose, croscarmellose sodium and magnesium stearate. The mixture obtained is compressed to obtain 300 mg tablets.

### EXAMPLE 3

Complexed Itraconazole Comprising Excipients in Capsules

20 g of succinic acid, 100 g of itraconazole, 20 g of a copolymer of a polyoxyethylenepolyoxypropylene-blockpolymer (Pluronic F 127) and 20 g of hydroxypropylmethyl-cellulose (E 5) are dissolved under stirring in a mixture of methanol and dichloromethane. 100 g of powdered potassium dihydrogen phosphate and 50 g of lactose are suspendend in the solution obtained. The solvent from the mixture obtained is evaporated off (dryness). The solid residue obtained is grinded to obtain a pre-mix. The X-ray powder diffraction pattern and the Raman spectrum of said pre-mix are shown in FIGS. 4 and 5.

The pre-mix obtained is blended with 192 g of lactose and 2.5 g of magnesium stearate.

The blend obtained is filled into hard gelatine capsule such, that one capsule contains the equivalent of 100 mg of itraconazole.

The melting point of itraconazole in the pre-mix is 154° C., whereas the melting point of pure itraconazole is determined to be 168° C.

### EXAMPLE 4

Complexed Itraconazole Comprising Excipients in Capsules

16.7 g of succinic acid, 100 g of itraconazole and 20 g of a copolymer of a polyoxyethylenepolyoxypropylene-blockpolymer (Pluronic F 127) are dissolved under stirring in a mixture of methanol and dichloromethane. A solution obtained is spray-dried using a spray drying equipment under nitrogen with an inlet temperature of 1100 and an outlet temperature of 500. The dry powder obtained is blended with lactose and magnesium stearate and the blend obtained is filled into hard gelatine capsules.

### EXAMPLE 5

Complexed Itraconazole Comprising Excipients in Capsules

20 g of succinic acid, 100 g of itraconazole and 20 g of hydroxypropylmethylcellulose (E 5) are dissolved under stirring in a mixture of methanol and dichloromethane and 100 g of powdered potassium dihydrogen phosphate are suspendend in the solution obtained. The solvent of the suspension obtained is evaporated off (dryness) and the solid residue is grinded and blended with 180 g of lactose and 2.5 g of magnesium stearate. The blend obtained is filled into hard gelatine capsule such, that one capsule contains the equivalent of 100 mg of itraconazole.

### EXAMPLE 6

Itraconazole Complex

Method A

30.3 mg succinic acid and 301.8 mg of itraconazole are dissolved in a mixture of methanol:dichloromethane 1:1. The solvent of the solution obtained is removed without vacuum. A solid is obtained.

Method A

A mixture comprising 7.7% (w/w) of succinic acid and 96.3% (w/w) of itraconazole is grinded and the mixture obtained is melted. The melt obtained is cooled quickly. On re-heating the mixture to 143° C. re-crystallisation occurs.

According to both methods A and B a new molecule is obtained having an X-ray powder diffraction pattern diagram as shown in FIG. 2 and a Raman spectrum as shown in FIG. 3. That X-ray powder diffraction pattern diagram as shown in FIG. 2 and that Raman spectrum as shown in FIG. 3 both are different from the X-ray powder diffraction pattern diagram, or the Raman spectrum, respectively, of itraconazole. The new molecule obtained is believed to be a complex between itraconazole and succinic acid. From the amounts of itraconazole and succinic acid used a ratio of itraconazole:succinic acid in said complex of 2:1 is expected.

### DESCRIPTION OF THE FIGURES

FIG. 1

Shows the release profile of a commercially available itraconazole formulation, namely Sporanox®, 100 mg capsules, Janssen Cilag B.V (-♦-) in comparison with a composition of the present invention, namely a pre-mix as described in example 3 (-▪-). The dissolution profile is determined in a dissolution test in 0.1N HCl containing 0.2% SLS. An US-type II (paddle) apparatus is used, speed 75 rpm, temperature 37±0.5° C. Samples are taken after 5, 10, 15, 30, 45 and 60 minutes. UV-detection at 258 nm.

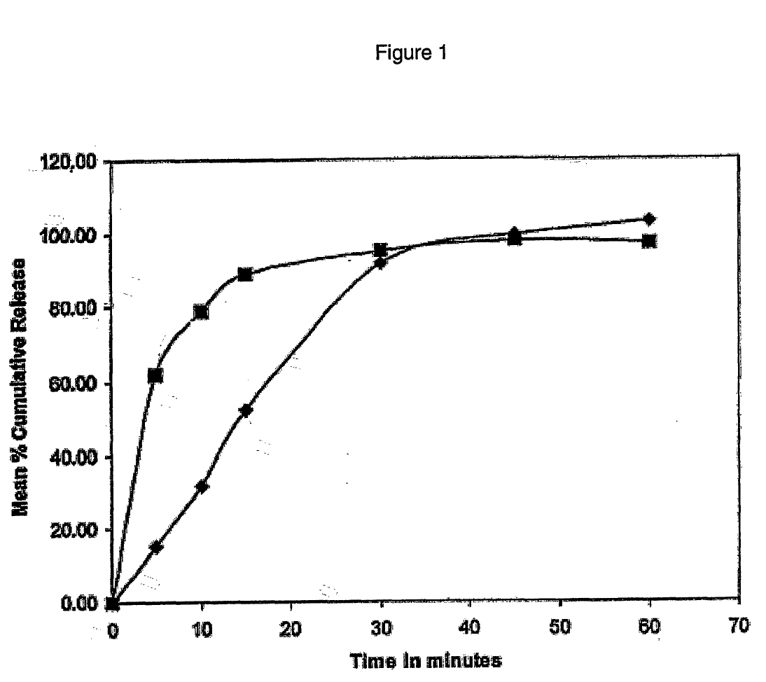

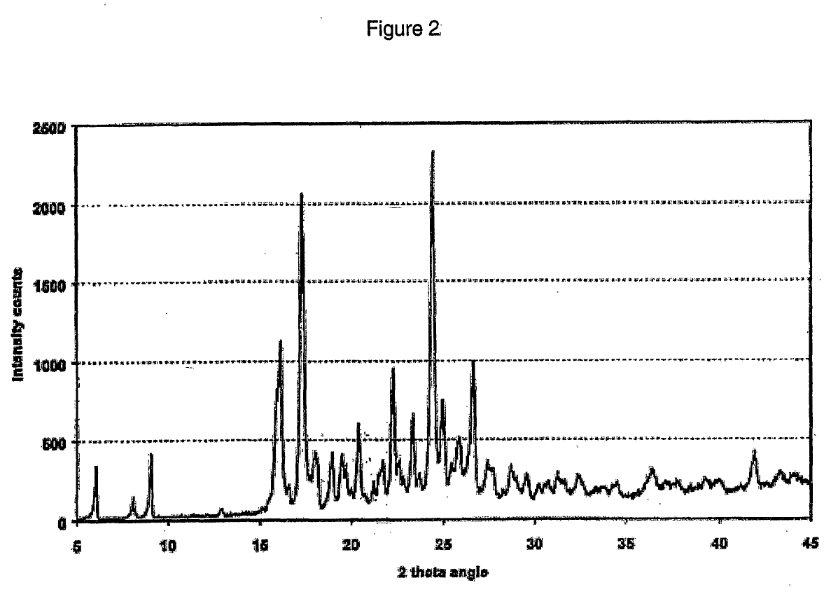

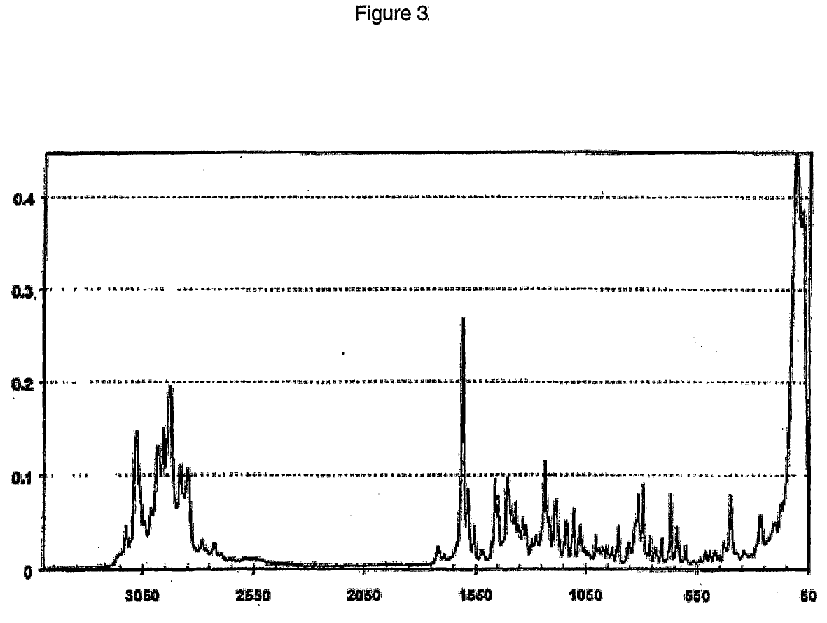

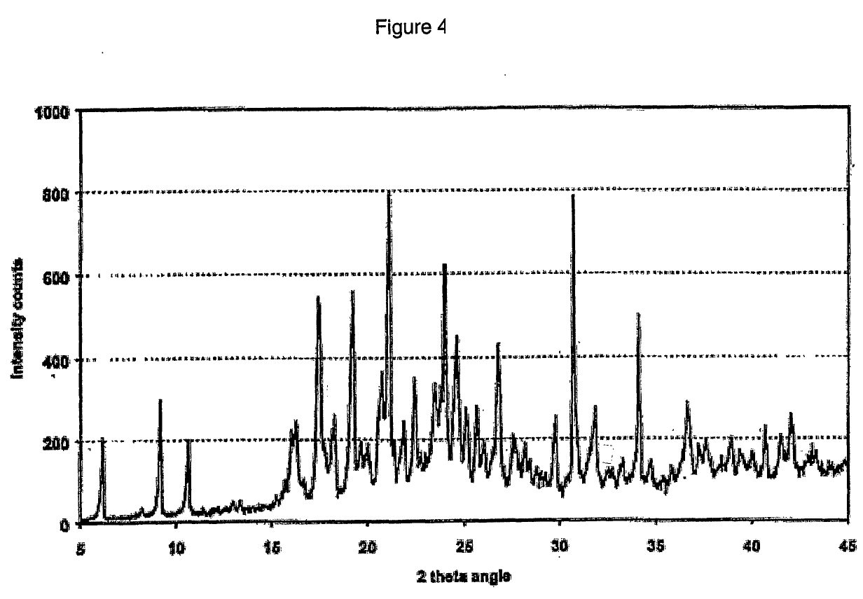

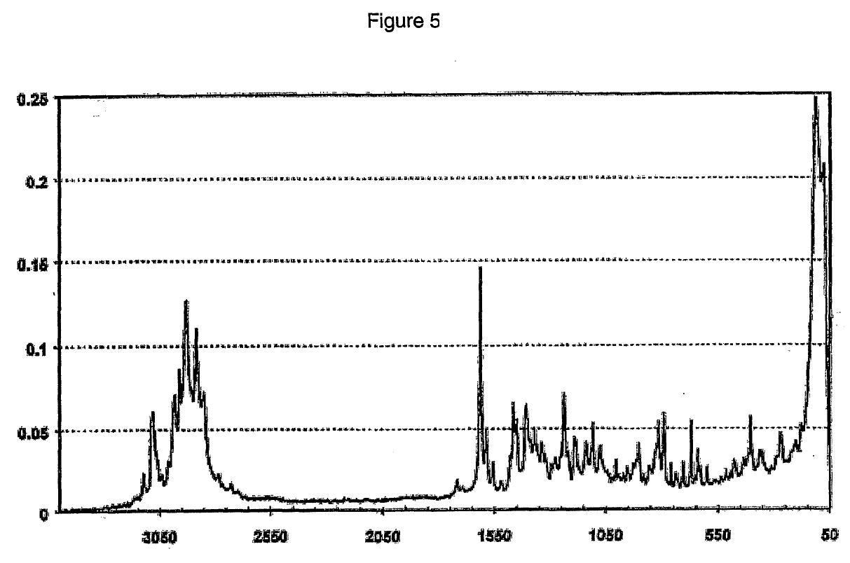

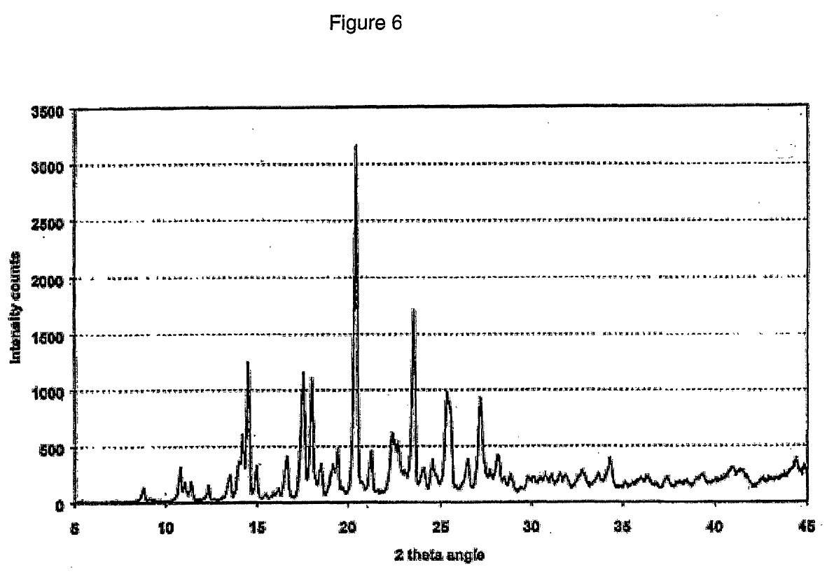

From FIG. 1 it is evident that the initial release rate of itraconazole from the commercially available itraconazole formulation Sporanox® is much slower than the release rate of a composition according to the present invention. E.g. after 5 minutes about 18%, after 10 minutes about 30% and after 15 minutes about 50% itraconazole are released from Sporanox®, whereas after 5 minutes more than 60%, after 10 minutes about 80% and after 15 minutes about about 90% of the itraconazole are released from a composition according to the present invention.

FIG. 2

Shows the X-ray powder diffraction pattern diagram of a complex of itraconazole:and succinic acid obtained according to example 6.

FIG. 3

Shows the Raman spectrum of a complex of itraconazole:and succinic acid obtained according to example 6.

FIG. 4

Shows the X-ray powder diffraction pattern diagram of a composition according to the present invention which is a pre-mix obtained according to Example 3.

FIG. 5

Shows the Raman spectrum of a composition according to the present invention which is a pre-mix obtained according to Example 3. A comparison of the Raman spectrum of FIG. 5 with the Raman spectrum of itraconazole shows practically no itraconazole in the original, uncomplexed form.

FIG. 6

Shows the X-ray powder diffraction pattern diagram of itraconazole.

A comparison of FIG. 4 and FIG. 6 shows that in the X-ray powder diffraction pattern diagram o a pre-mix obtained according to Example 3 there are only traces of itraconazole in the original, uncomplexed form.

In FIGS. 3 and 5 the vertical axis in the diagrams indicates Raman units and the horizontal axis in the diagrams indicates wave numbers (reciprocal wavelength in cm⁻¹).

1. A composition comprising itraconazole and an organic acid with the proviso that
formulations for oral administration comprising an antifungal, a sufficient amount of a cyclodextrin or a derivative thereof, an aqueous acidic medium as a bulk liquid carrier and an alcoholic co-solvent,
multicomponent inclusion complexes characterized in that a basic-type drug (guest molecule), an acid and a cyclodextrin are present,
solid dispersed preparations for poorly water-soluble drugs, prepared by dissolving or dispersing the drug in anoil, a fatty acid or a mixture thereof, mixing the solution or dispersion in a water-soluble polyol matrix and drying the mixture,
pharmaceutical compositions comprising a no more than sparingly water-soluble drug compound, a cyclodextin, a physiologically tolerable water-soluble acid, and a physiologically tolerable water-soluble organic polymer, and
pharmaceutical water-soluble formulations with a content on the phosphodiesterase (PDE)-type-5-inhibitor sildenafil or pharmaceutically acceptable salts thereof in combination with itraconazole and comprising citric, ascorbic and/or tartaric acid; are excluded.

2. A composition comprising itraconazole and an organic acid, wherein chemical and/or physical characteristics of itraconazole are different from chemical and/or physical characteristics of pure itraconazole.

3. Itraconazole in the form of a complex with an organic acid.

4. A composition according to any one of claims 1 to 3 and further comprising at least one excipient.

5. A composition according to any one of claims 1 to 4, wherein itroconazole has a dissolution rate in the USP paddle test, 75 rpm, 1000 ml 0.1N HCl+0.2% SLS, UV detection at 258 nm; which is after 10 minutes faster than the dissolution rate of itraconazole in Sporanox® formulations.

6. A composition according to any one of claims 1 to 5, wherein itroconazole has a dissolution rate in the USP paddle test, 75 rpm, 1000 ml 0.1N HCl+0.2% SLS, UV detection at 258 nm a dissolution rate which is
after 5 minutes of about 55% to 65%, such as around 60%; and/or
after 10 minutes of about 75% to 85%, e.g. around 80%; and/or;
after 15 minutes of about 86% to 92%, e.g. around 90%.

7. Use use of a composition according to any one of claims 1 to 6 in the production of a pharmaceutical composition comprising itraconazole as an active ingredient.

8. A pharmaceutical composition comprising itraconazole and a pharmaceutically acceptable organic acid according to any one of claims 1 to 6 beside at least one pharmaceutical carrier or diluent.

9. A pharmaceutical composition according to claim 8 wherein the pharmaceutical carrier or diluent comprises lubricant, and/or a binder, and/or filler, and or disintegrant, and/or surfactant; and/or hydrocolloid, and/or buffer substance.

10. A composition according to any one of claims 2 to 9 wherein the organic acid is succinic acid or maleic acid.
